(19) United States
(12) Patent Application Publication
(10) Pub. No.: US 2022/0122743 A1
(43) Pub. Date: Apr. 21, 2022

(54) RAPID HIGH-CAPACITY POPULATION SCREENING

(71) Applicant: MX3 Diagnostics, Inc., Austin, TX (US)

(72) Inventors: Michael Erlichster, Caulfield North (AU); Gursharan Chana, Fitroy North (AU); Efstratios Skafidas, Thornbury (AU)

(21) Appl. No.: 17/323,058

(22) Filed: May 18, 2021

Related U.S. Application Data
(60) Provisional application No. 63/026,971, filed on May 19, 2020.

(51) Int. Cl. G16H 70/60; G16H 50/80; G16H 50/30

(52) U.S. Cl. CPC G16H 70/60; G16H 50/30; G16H 50/80

(57) ABSTRACT

A method and system improving nucleic-acid testing-capacity for novel pathogens by applying family-level nucleic acid testing. The test system involves the use of an analyzer platform and single-use test cartridges which can identify the presence of one or multiple pathogens on the family-level. The test system is pro-actively stockpiled to ensure widespread availability for population screening in the event of a pandemic.

### CROSS-REFERENCE TO RELATED APPLICATIONS

This application claims the benefit of U.S. Provisional Patent Application No. 63/026,971, filed May 19, 2020, entitled, “RAPID HIGH-CAPACITY POPULATION SCREENING.” The disclosure of this priority application is hereby incorporated by reference in its entirety into the present application.

### TECHNICAL FIELD

This application describes biomedical systems and methods. More specifically, the application describes a method and system for applying nucleic acid testing with family level specificity to provide high-capacity population screening of novel pathogens shortly after pathogen discovery.

### BACKGROUND

During the early stages of the severe acute respiratory syndrome coronavirus 2 (SARS-CoV-2) pandemic, many countries exhibited an extreme shortage of SARS-CoV-2 nucleic acid test kits. This resulted in a month-long period where testing could only be performed in a limited capacity at select test centers, hampering efforts to control disease spread. The cause of this shortage was a combination of unprecedented global demand for test reagents and equipment, disruption to supply chains caused by the pandemic, and regulatory restrictions limiting the ability of some nations to expand test capacity. These shortcomings highlight that the current testing infrastructure and ability to expand testing capacity are not rapid enough to counter disease spread during the early stages of some pandemics.

While improvements in capacity and distribution of testing equipment and greater stockpiles of reagents may enhance the speed at which testing capacity may be increased during future pandemics, there will still be a vital period between when a novel pathogen emerges and when tests are widely available at high capacity, thus hampering efforts to contain disease spread. The length of this period is defined by several key milestones:

Milestone 1: Pathogen isolation and characterization. The novel pathogen is identified, and its genome is sequenced using complex and sophisticated laboratory techniques. This was relatively quick for the current SARS-CoV-2 pandemic, as expert infectious disease laboratories were present within the site of outbreak in Wuhan, but this is likely not always going to be the case.

Milestone 2: A bespoke nucleic acid test is developed and validated by government laboratories (e.g., the CDC). Testing is only available at select public laboratories, with a highly restricted capacity (100 s to 1000 s of tests per day).

Milestone 3: Industry partners mass produce government designed tests, or develop alternative tests, greatly increasing test capacity. Unrestricted testing is available at many different locations. (Millions of tests per day).

Depending on the availability of resources and stringency of regulations, it may take weeks or months to meet these three milestones. Additionally, the rushed timeline for test development and production may result in insufficient quality control and regulatory validations of tests, resulting in inaccurate tests and further delays. Also, during routine pathogen testing, disruptions to testing capacity may occur due to pathogen mutation(s) rendering species-specific testing inaccurate. Disruption may also occur due to supply chain or quality control issues.

Therefore, it would be desirable to have a method and system that provide for wide-spread testing of novel pathogens in a more timely manner, with a high level of quality control and regulatory certification. It would also be desirable to have a method and system that may be used to bridge temporary deficiencies in testing capacity. Ideally, such a method and system would be small and portable, allowing for testing to be performed at a well distributed range of sites, such as ports of entry. The system and method should also be rapid, allowing for high throughput screening of large populations, and cost effective, to make it accessible to many users across economic and geographical barriers. This application addresses at least some of these objectives.

### SUMMARY

The present application describes a system and method for rapid-availability, high-capacity, nucleic acid testing of novel pathogens. This application also describes a method for applying family-level nucleic acid tests for rapid pathogen identification and to act as an auxiliary tool to bridge disruptions in routine pathogen testing. Having a multiple family test will provide infectious disease laboratories with key information as to pathogen type to focus their research efforts.

These and other aspects and embodiments are described in greater detail below, in relation to the attached drawing figures.

### BRIEF DESCRIPTION OF THE DRAWINGS

FIG. 1A shows a system for measuring the presence of a pathogen in a sample, using pan-family assays, in which one cartridge is used is used to determine the presence of one viral family and multiple cartridges are available for use with a single analyzer, according to one embodiment;

FIG. 1B shows a system for measuring the presence of a pathogen in a sample, using pan-family assays, in which one cartridge is used to test for the presence of multiple viral families with a single analyzer, according to one embodiment;

FIG. 1C shows a system for measuring the presence of a pathogen in a sample, using pan-family assays, in which test reagents which are common for multiple test cartridges are stored on a separate sub-cartridge and the reagent cartridge is combined with the family-specific cartridge prior to insertion into the analyzer, according to one embodiment;

FIG. 2 is a flow chart demonstrating how the proposed method of testing differs from the currently available method, according to one embodiment; and

FIGS. 3A-3B are a set of tables illustrating how genomic sequence data (FIG. 3A) and observed protein data (FIG. 3B) can be used to derive a target sequence for a pan-family assay.

### DETAILED DESCRIPTION

The present application describes various embodiments and features of a system and method for rapid high-capacity testing of a novel pathogen. Although the following disclosure focuses on the analysis of saliva or blood, the embodiments described below, or variations of those embodiments, may be used for analysis of any other sample such as urine, fecal matter or sweat.

The Pan-Family Assay

A pan-family assay is a molecular diagnostic test capable of identify all organisms of within a family of species (e.g., a family of viruses such as Coronaviridae). Pan family tests are targeted at highly conserved genomic regions, allowing for identification of novel species, for which genomic sequence data is not yet available. Pan-family assays targeting several viral families have been developed for research applications and are routine used during pathogen characterization and in retrospective epidemiological studies. Currently, pan-family assays are not routinely used in clinical settings as they are not considered to be sufficiently specific.

A panel of pan-family assays would be required for a robust tool for combating novel pathogens. Pan-family assays have been described for multiple viral families associated with recent epidemics, including coronaviruses (SARS-CoV, MERS, SARS-CoV-2) filoviruses (Ebola Virus) and flaviviruses (Zika Virus, Dengue Virus, Yellow Fever Virus.

The Test System

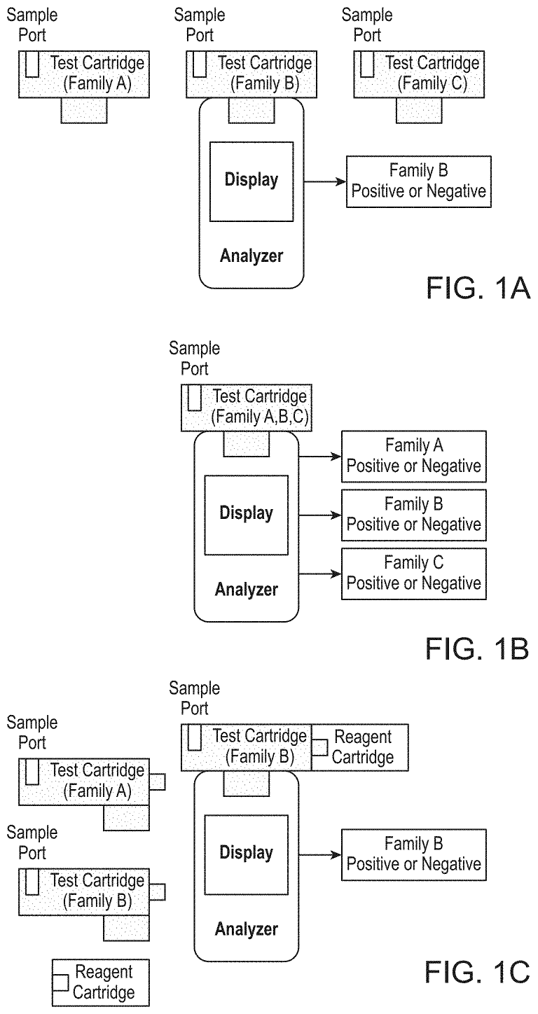

FIGS. 1A-1C show multiple embodiments of a test system capable of determining the presence of pathogens with a family level of specificity within a patient sample. These pathogens may include but are not limited to viruses (e.g., coronaviruses, filoviruses, flavivirus, influenza viruses), bacteria, fungi and prions. The nucleic acid amplification technique used to identify pathogens may include, but are not limited to, Polymerase Chain Reaction (PCR), Loop-Mediate Isothermal Amplification (LAMP) and ion semiconductor detection of DNA polymerization.

The test system may be a benchtop system or may be a handheld portable device. The test system may be able to communicate with a data tracking database. In one embodiment (FIG. 1A), a single-use test cartridge containing test reagents for a single viral family is inserted into a handheld analysis system to perform a test, with multiple different cartridges available to perform tests for different viral families. In another embodiment (FIG. 1B), the single use test cartridge contains test reagents for multiplexed testing of multiple viral families at the same time. In another embodiment (FIG. 1C) a secondary reagent test cartridge in inserted into the main cartridge containing reagents specific to a viral family prior to insertion into the analysis system, allowing for more efficient use of stockpiled reagents.

The Test Cartridge

Tests cartridges may be used to collect a sample directly from an orifice (e.g., saliva from the mouth), or from a collection receptacle (e.g., a blood samples). Sample processing may be required prior to insertion into the test cartridge (e.g., mixing of a patient sample with a buffer) or processing may be automated and occur within the test cartridge.

Test cartridges are designed for ease of use to minimize exposure to patient samples, reduce hands-on time as well as allow testing with minimal training or facility certification.

Test cartridges may also include additional tests targeting common pathogens (e.g., common human coronavirus species). This additional test may improve the specificity of a test for a novel pathogen.

The Pan-Family Stockpile Method

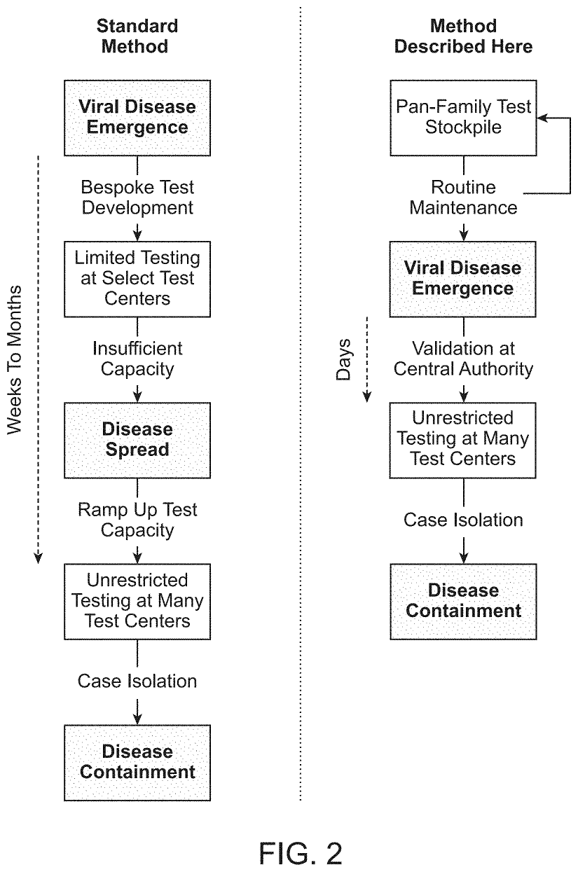

Large-scale stockpiling of the Test System and Test Cartridges is employed at multiple locations to reduce the likelihood of severe test shortages in future pandemics. In contrast to the traditional test capacity building procedures, this allows for more rapid, extensive and distributed testing (FIG. 2).

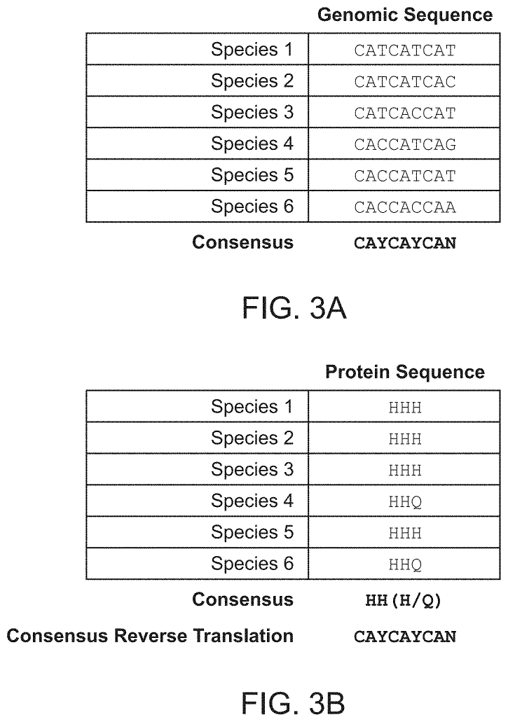

In this method several pan-family tests are developed, certified and stockpiled in large volume prior to disease emergence. Target sequences of pan-family tests may be designed using available sequence data (FIG. 3A) or using available protein data to predict genomic data (FIG. 3B). As pan-family assays are developed prior to immediate need, they have a relaxed timeframe for test development, ensuring best-practice validation, quality control procedures, regulatory certification and laboratory accreditation can be achieved.

Ongoing maintenance of stockpiled cartridges and analyzers is performed to ensure test accuracy. Routine monitoring of novel species reported in animal populations is performed to ensure assay design effectively captures observed variation, and test cartridges are modified to reflect novel variation.

Upon the emergence of a novel pathogen, a centralized laboratory runs a series of tests to determine the sensitivity of the stockpiled test cartridges to the novel pathogen. Following confirmation of test cartridges applicability to the novel pathogen, the test cartridges are approved for use.

Test cartridges and analyzers are then applied to deliver unrestricted testing at many testing facilities while a species-specific test is produced. Test results may be used to directly guide intervention measures (e.g., isolation and/or treatment of infected individuals) or used to guide application of short-supply species-specific tests.

By eliminating the need to mass produce and distribute tests after a pathogen is identified, this strategy may greatly reduce the time between pathogen identification and unrestricted testing compared to traditional capacity building strategies.

The described test system will also be used as an auxiliary tool to boost testing capacity during routine pathogen testing if there is a disruption limiting species-specific test availability. Such disruptions may include mutations reducing the accuracy of specific tests, disruptions to supply chains of test reagents or disruptions to the accuracy of testing due to quality control issues.

We claim:

1. A system for determining the presence of a novel pathogen in a patient sample using pan-family nucleic acid testing, the system comprising:
a single-use reagent cartridge configured to detect the presence of pathogens on the family level; and
an analyzer device configured to facilitate testing.

2. The system of claim 1, wherein the analyzer is a portable handheld device.

3. The system of claim 1 wherein the analyzer performs automated result analysis and interpretation.

4. The system of claim 1, wherein the analyzer contains a wireless communication chip allowing for over-the-air transfer of calibration data or test results.

5. The system of claim 1, wherein the reagent cartridge is used to directly collect the patient sample before inserting into the analyzer.

6. The system of claim 1, wherein a patient sample is collected in a receptacle and then transferred to the reagent cartridge.

7. The system of claim 1, wherein the reagent cartridge contains a single test targeted at a family of pathogens.

8. The system of claim 1, wherein the reagent cartridge contains multiple tests which are targeted at multiple families of pathogens.

9. The system of claim 1, wherein the reagent cartridge contains additional test targeted at known pathogens to improve test specificity.

10. The system of claim 1, wherein reagents common between multiple test cartridges are stored in a sub-cartridge which is inserted into the reagent cartridge before use.

11. A method for rapidly screening large populations for a novel pathogen using the system of claim 1, the method comprising:
developing reagent cartridges prior to a pandemic using observed pathogen variation;
stockpiling reagent cartridges and analyzers at multiple testing facilities;
maintaining reagent cartridges over a period of time;
validating cartridge sensitivity to a novel pathogen; and
using the stockpiled cartridges and analyzers to perform testing for the novel pathogen.

12. The method of claim 11, wherein the reagent cartridges are developed using observed genomic sequence data from species within a given pathogen family.

13. The method of claim 11, wherein the reagent cartridges are developed using genomic sequence data predicted from observed protein sequences within a given pathogen family.

14. The method of claim 11, wherein maintaining the reagent cartridges comprises making a routine comparison of reagent cartridges with novel variation observed in non-human populations.

15. The method of claim 11, wherein validating cartridge sensitivity is performed by a central authority to confirm cartridge sensitivity for a novel pathogen prior to wide-scale application.

16. The method of claim 11, wherein the test result is used to isolate infected individuals to prevent disease transmission.

17. The method of claim 11, wherein the test result is used to direct the application of high-specificity confirmatory testing for the novel pathogen.

18. The method of claim 11, wherein the test result is used to direct the application of high-specificity confirmatory testing for a panel of known pathogens.

19. A method for preventing disruptions to nucleic acid testing capacity using the system of claim 1, method comprising:
developing reagent cartridges using observed pathogen variation;
large scale stockpiling of reagent cartridges and analyzers at many testing facilities;
ongoing maintenance and validation of test cartridges; and
application of stockpiled cartridges and analyzers to perform testing for the novel pathogen during disruption to regular testing methods.

20. The method of claim 19, wherein the disruption to testing capacity is caused by pathogen mutation interfering with the sensitivity of species-specific testing.

21. The method of claim 19, wherein the disruption to testing capacity is caused by supply chain issues.

22. The method of claim 19, wherein the disruption to testing capacity is caused by quality control issues.

23. The method of claim 19, wherein the ongoing maintenance and validation of test cartridges involves routine observation of pathogen mutation and validation of test cartridge sensitivity in the presence of observed mutations by a central authority.
